(19) United States
(12) Patent Application Publication
(10) Pub. No.: US 2022/0403127 A1
(43) Pub. Date: Dec. 22, 2022

(54) MIXED-CHARGE COPOLYMER ANTIBIOFILM COATINGS

(71) Applicant: Nanyang Technological University, Singapore (SG)

(72) Inventors: Bee Eng Mary Chan, Singapore (SG); Yang WU, Singapore (SG); Zheng Hou, Singapore (SG)

(21) Appl. No.: 17/787,559

(22) PCT Filed: Jan. 11, 2021
(86) PCT No.: PCT/SG2021/050018

§ 371 Date: Jun. 20, 2022

(30) Foreign Application Priority Data
Jan. 9, 2020 (SG) 10202000204Y

(51) Int. Cl. C08J 7/16; C08F 220/60; C08F 220/38; C08F 220/34; A61L 29/12; A61L 29/16

(52) U.S. Cl. CPC C08J 7/16; C08F 220/606; C08F 220/38; C08F 220/34; A61L 29/126; A61L 29/16; A61L 2300/404; C08J 2375/04

(57) ABSTRACT

Disclosed herein is a composite material suitable for inhibiting biofilm growth, the composite material comprising a substrate material and a random copolymeric material covalently bonded to a surface of the substrate material. The random copolymer contains repeating units having at least one functional group bearing a cationic charge and repeating units having at least one functional group bearing an anionic charge, where the repeating units are derived from compatible monomers that belong to different monomer classes having differing polymerisation kinetics. Specifically, the random copolymeric material is poly(AMPTMA-ran-SPM), wherein AMPTMA is (3-acrylamidopropyl) trimethylammonium chloride and SPM is 3-sulfopropyl methacrylate potassium. Also disclosed are methods of manufacturing said material and applications thereof.

### FIELD OF INVENTION

The current invention relates to mixed-charge copolymer antibiofilm coatings, as well as methods of formation and use thereof.

### BACKGROUND

The listing or discussion of a prior-published document in this specification should not necessarily be taken as an acknowledgement that the document is part of the state of the art or is common general knowledge.

Medical catheters are used for a myriad of modern medical interventions, such as draining urine and intravenous drug delivery. However, catheter-related infections are common because indwelling catheters are prone to bacterial contamination. Several measures, such as shortening the implantation period, sterilization of the surgical environment and flushing of lumen with an antibiotic stock solution periodically, have been taken to overcome biofilm formation on catheters. However, these measures have proven to be ineffective for indwelling catheters. In addition, despite the implementation of these measures, the incidence of catheter-related infections (CRIs) have not decreased and new strategies are urgently needed.

The development of catheter surface coatings is one strategy to combat CRIs. Catheter surface coatings can be classified into two designs, namely leachable and non-leachable. Leachable catheters entail the incorporation of leachable bactericidal agents into the coating or the catheter itself, and these agents can actively kill the bacteria as they leach out. However, sustained release and toxicity issues associated with leachable coatings remain unsolved. Another class of antibacterial coatings are non-leachable hydrophilic coatings. Most commercially available non-leachable hydrophilic coatings are made from non-charged hydrophilic polymers such as polyvinylpyrrolidone or polyacrylamide. Besides these polymers, hydrophilic non-fouling coatings based on zwitterionic polymers have also been widely explored. Zwitterionic polymers can be made from integrated zwitterion monomers, whereby each monomer has both a cationic and an anionic charge or from a pair of mixed-charge monomers where cationic monomers are mixed with anionic monomers. For integrated zwitterionic coatings, water molecules are able to form a hydration layer by electrostatic interaction with the integrated zwitterionic surface, making the surface stealthy and non-fouling.

However, integrated zwitterionic monomers usually have high viscosity due to their poor solubility in pure water and are relatively more expensive than other options.

In addition, mixed-charge zwitterionic polymer coatings can also make surfaces highly hydrophilic but they are generally much less explored.

Thus, there remains a need to develop new polymer coatings that are affordable and can overcome the problems associated with conventional CRIs.

### SUMMARY OF INVENTION

It has been surprisingly found that certain mixed-charge zwitterionic polymer coatings have superior properties. Aspects and embodiments of the invention are set forth in the following numbered clauses.

1. A composite material suitable for inhibiting biofilm growth, the composite material comprising:
a substrate material; and
a random copolymeric material covalently bonded to a surface of the substrate material, wherein the random copolymeric material has a formula I:

[E]-[G] I

where:
E represents a repeating unit derived from a monomer J, where the monomer J has at least one functional group bearing a cationic charge; and
G represents a repeating unit derived from a monomer L, where the monomer L has at least one functional group bearing an anionic charge, wherein:
the monomers J and L belong to different, but compatible, monomer classes with differing polymerisation kinetics.

2. The composite material according to Clause 1, wherein the monomer classes are vinyl monomers selected from vinyl benzyl monomers and vinyl benzene monomers or, more particularly, acrylamide, alkylacrylamide, acrylate and alkylacrylate, optionally wherein the monomer classes are acrylamide, acrylate and methacrylate.

3. The composite material according to Clause 1 or Clause 2, wherein the random copolymeric material is selected from:

(Ai) poly(cationic acrylamide-ran-anionic methacrylate);
(Aii) poly(cationic acrylamide-ran-anionic acrylate);
(Aiii) poly(cationic acrylamide-ran-anionic methacrylamide);
(Aiv) poly(cationic acrylamide-ran-anionic vinyl monomer)
(Av) poly(cationic methacrylate-ran-anionic acrylate);
(Avi) poly(cationic methacrylate-ran-anionic acrylamide);
(Avii) poly(cationic methacrylate-ran-anionic methacrylamide);
(Aiii) poly(cationic methacrylate-ran-anionic vinyl monomer)
(Aix) poly(cationic acrylate-ran-anionic methacrylate);
(Ax) poly(cationic acrylate-ran-anionic acrylamide);
(Axi) poly(cationic acrylate-ran-anionic methacrylamide);
(Axii) poly(cationic acrylate-ran-anionic vinyl monomer);
(Axiii) poly(cationic vinyl monomer-ran-anionic methacrylate);
(Axiv) poly(cationic vinyl monomer-ran-anionic acrylate);
(Av) poly(cationic vinyl monomer-ran-anionic acrylamide);
(Avi) poly(cationic vinyl monomer-ran-anionic methacrylamide),
optionally wherein, the random copolymeric material is a poly(cationic acrylamide-ran-anionic methacrylate).

4. The composite material according to any one of the preceding clauses, wherein the at least one functional group bearing an anionic charge in monomer L is selected from one or more of the group consisting of sulfonic, carboxylate, and phosphate.

5. The composite material according to Clause 4, wherein the at least one functional group in monomer L is a sulfonic functional group.

6. The composite material according to any one of the preceding clauses, wherein the at least one functional group bearing a cationic charge in monomer J is selected from one or more of the group consisting of phosphonium, imidazolium, and ammonium.

7. The composite material according to Clause 6, wherein the at least one functional group in monomer J is an ammonium functional group.

8. The composite material according to any one of Clauses 1 to 6, wherein monomer J is selected from (3-acrylamidopropyl) trimethylammonium chloride (AMPTMA), [2-(methacryloyloxy) ethyl] trimethylammonium chloride (MAETMA), [2-(acryloyloxy) ethyl] trimethylammonium chloride (AETMA), [3-(methacryloylamino)propyl] trimethylammonium chloride (MAPTAC), (3-acrylamidoethyl) methylimidazolium chloride (AMEMI), [2-(methacryloyloxy) ethyl] methylimidazolium chloride (MAEMI), [2-(acryloyloxy) ethyl] methylimidazolium chloride (AEMI), [3-(methacryloylamino)ethyl] methylimidazolium chloride (MAAEMI), (vinylbenzyl) trimethylphosphonium chloride (VBTMP), and (vinylbenzyl) trimethylammonium chloride (VBTMA).

9. The composite material according to any one of Clauses 1 to 4 and any one of Clauses 6 to 8 as dependent upon any one of Clauses 1 to 4, wherein monomer L is selected from 3-sulfopropyl methacrylate potassium (SPM), 3-sulfopropyl acrylate potassium (SPA), 2-acrylamido-2-methylpropane sulfonate sodium (AMPA), 3-sulfopropyl methacrylamide potassium (SPMA), 2-carboxyethyl acrylate (CEA), 2-carboxyethyl methacrylate (CEM), 2-carboxylethyl acrylamide (CEAM), 2-carboxylethyl methacrylamide (CEMA), sodium 4-vinyl benzenesulfonate, and 4-vinylbenzoic acid.

10. The composite material according to Clause 9, wherein monomer L is selected from 3-sulfopropyl methacrylate potassium (SPM), 3-sulfopropyl acrylate potassium (SPA), and 2-acrylamido-2-methylpropane sulfonate sodium (AMPA).

11. The composite material according to any one of the preceding clauses, wherein the random copolymeric material is selected from:

(ai) poly(AMPTMA-ran-SPM);
(aii) poly(AMPTMA-ran-SPA);
(aiii) poly(AETMA-ran-SPM);
(aiv) poly(AETMA-ran-AMPA);
(av) poly(MAETMA-ran-SPA); or
(avi) poly(MAETMA-ran-AMPA).

12. The composite material according to Clause 11, wherein the random copolymeric material is poly(AMPTMA-ran-SPM).

13. The composite material according to any one of the preceding clauses, wherein the substrate material is a polyurethane.

14. The composite material according to any one of the preceding clauses, wherein the composite material has a water contact angle of less than 50°, such as less than 40°, such as from 20° to 30°.

15. An article comprising a composite material as described in any one of Clauses 1 to 14.

16. The article according to Clause 15, wherein the article is a catheter.

17. A method of forming a composite material as described in any one of Clauses 1 to 14, the method comprising the steps of:

(aa) providing a mixture comprising a substrate material, a solvent, a monomer J that has at least one functional group that bears a cationic charge, and a monomer L that has at least one functional group that bears an anionic charge, where the monomers J and L are monomers that are compatible to form a random copolymer; and
(ab) adding an initiator to the mixture to form a reaction mixture and allowing the reaction mixture to age for a period of time to provide the composite material, wherein
the monomers J and L belong to different monomer classes with differing polymerisation kinetics.

### DRAWINGS

FIG. 1 shows the surface composition of poly(AMPTMA-ran-SPM) (a) FTIR-ATR spectra, XPS (b) wide scan, (c) N1s scan and (d) S2p scan spectra of: (i) PU; (ii) PU/poly(AMPTMA-ran-SPM); (iii) PU/poly(AMPTMA); and (iv) PU/poly(SPM).

FIG. 2 depicts the FTIR-ATR of mixed-charge coating formulations (a) SPM series; (b) SPA series; and (c) AMPA series.

FIG. 3 depicts the XPS N1s (a-c) and S2p (d-f) of mixed-charge coating formulations.

FIG. 4 shows the (a) N⁺(CH₃)₃/SO₃⁻ molar ratio of poly(AMPTMA-ran-SPM) coating measured by angle-resolved XPS at 20°, 45°, 60°, 90° take-off angle respectively; (b) EDX mapping of N and S elements on poly(AMPTMA-ran-SPM) coated PU catheter surface. The scale bar is 10 μm; and (c) N⁺(CH₃)₃/SO₃⁻ molar ratio of mixed-charge copolymer coatings measured by XPS at 90° take-off angle; and (d) Hydrophilicity measured by water contract angle.

FIG. 5 shows that dimple structures are observed for the library of mixed-charge copolymer coatings by FESEM, the surface element is analysed by EDX mode. Scale bars are 10 μm.

FIG. 6 illustrates the water contact angle images of cationic and anionic homopolymers (a) poly(AMPTMA) (b) poly(AETMA) (c) poly(MAETMA) (d) poly(SPM) (e) poly(SPA) (f) poly(AMPA).

FIG. 7 shows the morphology features (a) FESEM morphology and (b) AFM topology images of (i) PU (ii) poly(AMPTMA-ran-SPM); Root-mean-square (rms) (c) roughness and (d) distance between adjacent peaks of mixed-charge copolymer coatings measured by AFM. Scale bars are 10 μm.

FIG. 8 shows the Zeta potential of mixed-charge coating formulations compared with that of unmodified PU catheter.

FIG. 9 depicts the redox polymerization kinetics in solution of cationic and anionic monomers (structures are summarized in FIG. 15). There is a break in the SPM curve due to the break in the Y axis.

FIG. 10 depicts the in vitro 24 hours' static antibiofilm efficacy of mixed-charge copolymer coatings against 6 types of bacteria: (a) Methicillin-resistant S. aureus BAA38 (MRSA); (b) Vancomycin-resistant E. faecalis V583; (c) Methicillin-resistant S. epidermidis 35984; (d) P. aeruginosa PAO1; (e) E. coli UT189; and (f) A. baumannii AB-1.

FIG. 11 illustrates the in vitro antibiofilm efficacy of poly(AMPTMA-ran-SPM) coating compared with controls (commercial silver catheter and poly(SBMA) coating) (a) 24 hours' static antibiofilm assay; (b) Live & Dead confocal microscopy of catheter incubated with Methicillin-resistant S. aureus BAA38 (MRSA) for 24 hours. Scale bars are 20 μm; (c) 24 hours' intraluminal dynamic antibiofilm assay; and (d) 30 days' static antibiofilm assay.

FIG. 12 shows the solution viscosity comparison between poly(SBMA) and poly(AMPTMA-ran-SPM) after 24 hours' polymerization.

FIG. 13 depicts the Biocompatibility assay with mammalian cells and blood cells (a) Cell viability of poly(AMPTMA-ran-SPM) coating measured by extraction method and MTT assay against 4 types of mammalian cells; (b) Protein fouling on different catheters. (c) Immune cell activation triggered by different catheters after 2 hours incubation with rabbit whole blood (control is 2nd bar from left for each group); (d) SEM images of thrombus coverage on catheter (I) PU; (II) silver catheter; (Ill) poly(SBMA) coated catheter; (IV) poly(AMPTMA-ran-SPM) coated catheter. Scale bars are 10 μm; and (e) Percentage of thrombus formed after 2 hours incubation in rabbit whole blood with catheters.

FIG. 14 illustrates the in vivo study of antibiofilm efficacies. Anti-biofilm efficacy of poly(AMPTMA-ran-SPM) coating against various bacteria in murine (a) urinary tract infection model and (b) subcutaneous wound infection model.

FIG. 15 O₃-activated redox graft copolymerization to form mixed-charge copolymers on polyurethane (PU) catheter surface.

### DESCRIPTION

It has been surprisingly found that a mixed-charge random copolymeric material can be used as a coating on a substrate to prevent the formation of biofilms of both Gram-positive and Gram-negative bacteria, while also being non-thrombogenic and non-toxic to mammalian cells.

In a first aspect of the invention, there is provided a composite material suitable for inhibiting biofilm growth, the composite material comprising:
a substrate material; and
a random copolymeric material covalently bonded to a surface of the substrate material, wherein the random copolymeric material has a formula I:

[E]-[G] I

where:
E represents a repeating unit derived from a monomer J, where the monomer J has at least one functional group bearing a cationic charge; and
G represents a repeating unit derived from a monomer L, where the monomer L has at least one functional group bearing an anionic charge, wherein:
the monomers J and L belong to different, but compatible, monomer classes with differing polymerisation kinetics.

The invention disclosed herein may also relate to a modified catheter comprising:
a) a catheter (e.g. polyurethane catheter)
b) a random copolymer on the surface of the catheter; the random copolymer comprising a mixed-charge pair of
   i. a cationic monomer; and
   ii. an anionic monomer.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

When used herein, the term “monomers J and L belong to different, but compatible, monomer classes with differing polymerisation kinetics” refers to materials that have a polymerisable group (e.g. carbon-to-carbon double bonds), but which may contain additional functionality that distinguishes them from one another with respect to polymerisation kinetics. For example, acrylate and methacrylate groups both contain a polymerisable vinylogous group, but differ in that one also contains a methyl group on the carbon-to-carbon double bond. This methyl group affects the polymerisation kinetics of the methacrylate, meaning that the methacrylate monomer has a slower rate of polymerisation than the acrylate monomer in the solution phase. Without wishing to be bound by theory, it is believed that the differing polymerisation kinetics of the monomers used to form the random copolymers used herein results in the improved properties disclosed herein. For example, the anionic monomer (SPM) having the slowest solution polymerization kinetics coupled with the cationic monomer (AMPTMA) having fastest solution polymerization kinetics resulted in a material displaying the highest anionic/cationic charge ratio of the materials tested herein. This material also displayed the most hydrophilic and most effective antibiofilm surface coating on catheters of the materials tested. Thus, it is believed that the relative kinetics of the anionic versus cationic monomer polymerization will affect the antibiofilm performance of the resulting mixed-charge copolymer coating.

Any suitably compatible monomers with differing polymerisation kinetics may be used to make the composite material disclosed herein. Suitable monomers that may be used include, but are not limited to vinyl monomers selected from vinyl benzyl monomers and vinyl benzene monomers or, more particularly, acrylamide, alkylacrylamide, acrylate and alkylacrylate, optionally wherein the monomers are acrylamide, acrylate and methacrylate.

Any suitable combination of monomers may be used to make the random copolymeric material used herein. For example, the random copolymeric material may be selected from:

(Ai) poly(cationic acrylamide-ran-anionic methacrylate);
(Aii) poly(cationic acrylamide-ran-anionic acrylate);
(Aiii) poly(cationic acrylamide-ran-anionic methacrylamide);
(Aiv) poly(cationic acrylamide-ran-anionic vinyl monomer)
(Av) poly(cationic methacrylate-ran-anionic acrylate);
(Avi) poly(cationic methacrylate-ran-anionic acrylamide);
(Avii) poly(cationic methacrylate-ran-anionic methacrylamide);
(Aiii) poly(cationic methacrylate-ran-anionic vinyl monomer)
(Aix) poly(cationic acrylate-ran-anionic methacrylate);
(Ax) poly(cationic acrylate-ran-anionic acrylamide);
(Axi) poly(cationic acrylate-ran-anionic methacrylamide);
(Axii) poly(cationic acrylate-ran-anionic vinyl monomer);
(Axiii) poly(cationic vinyl monomer-ran-anionic methacrylate);
(Axiv) poly(cationic vinyl monomer-ran-anionic acrylate);
(Av) poly(cationic vinyl monomer-ran-anionic acrylamide); or
(Avi) poly(cationic vinyl monomer-ran-anionic methacrylamide).

It will be appreciated that the term “ran” when used herein indicates a random copolymeric material.

In particular embodiments of the invention that may be mentioned herein, the random copolymeric material may be a poly(cationic acrylamide-ran-anionic methacrylate).

As will be appreciated, the monomers J and L contain further functional groups in their side-chains that are cationic and anionic, respectively.

Any suitable cationic functional group may be used in monomer J to provide the cationic charge. For example, the at least one functional group bearing a cationic charge in monomer J may be selected from one or more of the group consisting of phosphonium, imidazolium, and ammonium. In particular embodiments of the invention that may be mentioned herein, the at least one functional group in monomer J may be an ammonium functional group.

Any suitable anionic functional group may be used in monomer L to provide the anionic charge. For example, the at least one functional group bearing an anionic charge in monomer L may be selected from one or more of the group consisting of sulfonic, carboxylate, and phosphate. In particular embodiments of the invention that may be mentioned herein, the at least one functional group in monomer L may be a sulfonic functional group.

As will be appreciated, as the random copolymeric material is intended to be roughly zwitterionic, it may be the case that the number of cationic and anionic functional groups in J and L, respectively, are the same. Further, while the random copolymeric material is intended to be zwitterionic, there may be some fluctuation in the overall charge of the polymer, such that it may have an overall net positive charge, an overall net negative charge or an overall net neutral charge.

Suitable cationic monomers J may be selected from one or more of (3-acrylamidopropyl) trimethylammonium chloride (AMPTMA), [2-(methacryloyloxy) ethyl] trimethylammonium chloride (MAETMA), [2-(acryloyloxy) ethyl] trimethylammonium chloride (AETMA), [3-(methacryloylamino)propyl] trimethylammonium chloride (MAPTAC), (3-acrylamidoethyl) methylimidazolium chloride (AMEMI), [2-(methacryloyloxy) ethyl] methylimidazolium chloride (MAEMI), [2-(acryloyloxy) ethyl] methylimidazolium chloride (AEMI), [3-(methacryloylamino)ethyl] methylimidazolium chloride (MAAEMI), (vinylbenzyl) trimethylphosphonium chloride (VBTMP), and (vinylbenzyl) trimethylammonium chloride (VBTMA).

Suitable cationic monomers L may be selected from one or more of 3-sulfopropyl methacrylate potassium (SPM), 3-sulfopropyl acrylate potassium (SPA), 2-acrylamido-2-methylpropane sulfonate sodium (AMPA), 3-sulfopropyl methacrylamide potassium (SPMA), 2-carboxyethyl acrylate (CEA), 2-carboxyethyl methacrylate (CEM), 2-carboxylethyl acrylamide (CEAM), 2-carboxylethyl methacrylamide (CEMA), sodium 4-vinylbenzenesulfonate (VBS), and 4-vinylbenzoic acid (VBA). For example, L may be selected from one or more of 3-sulfopropyl methacrylate potassium (SPM), 3-sulfopropyl acrylate potassium (SPA), and 2-acrylamido-2-methylpropane sulfonate sodium (AMPA).

In embodiments of the invention, the random copolymeric material may be selected from:

(ai) poly(AMPTMA-ran-SPM);
(aii) poly(AMPTMA-ran-SPA);
(aiii) poly(AETMA-ran-SPM);
(aiv) poly(AETMA-ran-AMPA);
(av) poly(MAETMA-ran-SPA); or
(avi) poly(MAETMA-ran-AMPA).

For example, the random copolymeric material may be poly(AMPTMA-ran-SPM). The mixed-charge pair comprising the cationic (3-Acrylamidopropyl) trimethylammonium chloride (AMPTMA) and the anionic 3-Sulfopropyl methacrylate potassium salt (SPM) was found to be effective against biofilms of both Gram-positive and Gram-negative bacteria. Further, the AMPTMA/SPM coating is non-thrombogenic in rabbit blood and non-toxic to mammalian cells.

The AMPTMA/SPM coating results in the highest SO₃⁻/N⁺(CH₃)₃ molar ratio among the formulations tested herein. The anionic monomer (SPM) used has the slowest solution polymerization rate but the highest incorporation in the coating; the cationic monomer (AMPTMA) used has the fastest solution polymerization rate and the lowest coating incorporation. As discussed in the experimental section below, the good in vitro and in vivo antibiofilm performance of the AMPTMA/SPM coating may be attributed to the combination of hydrophilicity and anionic surface produced by the abundance of SO₃⁻. Compared with SBMA, the solution viscosity of AMPTMA/SPM after polymerization is much lower which makes the subsequent washing steps easy. It also has good biocompatibility with mammalian cells and rabbit blood. Hence, this new mixed-charge copolymer coating is a suitable candidate as antibiofilm coatings for catheters and possibly various medical catheters. It is believed that this study provides new insights into the design and optimization of mixed-charge copolymer coatings to achieve high hydrophilicity and good antibiofilm effects.

In embodiments of the invention that may be mentioned herein, the molar ratio of cationic/anionic materials may be from 0.2:1 to 1:1, such as from 0.3:1 to 0.8:1, such as from 0.4:1 to 0.6:1.

Any suitable material may be used as the substrate material, provided that it can form a covalent bond to the random copolymeric material. For example, the substrate material may be any suitably surface-activated material, such as a polymeric material, or a polymeric material that may form a covalent bond with the random copolymeric material without the need for surface activation. When used herein, surface activation may refer to the activation of a surface by any suitable treatment that enables the surface to form a covalent bond with the random copolymeric material (i.e. with a monomer used as the anchor from which the random copolymeric material grows from). Examples of suitable surface activation methods include, but are not limited to treatment with ozone or suitable plasmas, or surface-grafting of initiators. In embodiments of the invention that may be mentioned herein, the substrate material may be a polyurethane.

The composite material may have a hydrophilic surface. For example, the composite material may have a water contact angle of less than 50°, such as less than 40°, such as from 20° to 30°.

As will be appreciated, the composite material disclosed herein may itself be an article with particular applications, or it may be incorporated into such an article. Thus, in a further aspect of the invention, there is provided an article comprising a composite material as described in hereinbefore. For example, the substrate material may be a tube intended for use as a catheter, and so an article that may be formed from the composite material may be a catheter. It will be appreciated that the catheter may contain the coating on the outer and/or inner surface of the catheter. For example, both the inner and outer surfaces of the catheter tube may be coated with the random copolymeric material.

In a further aspect of the invention, there is also disclosed a method of forming a composite material as described hereinbefore, the method comprising the steps of:

(aa) providing a mixture comprising a substrate material, a solvent, a monomer J that has at least one functional group that bears a cationic charge, and a monomer L that has at least one functional group that bears an anionic charge, where the monomers J and L are monomers that are compatible to form a random copolymer; and
(ab) adding an initiator to the mixture to form a reaction mixture and allowing the reaction mixture to age for a period of time to provide the composite material, wherein
the monomers J and L belong to different monomer classes with differing polymerisation kinetics.

Further aspects and embodiments of the invention will now be discussed by reference to the following non-limiting examples.

### EXAMPLES

### Materials

Polyurethane (PU) catheters (Micro-Renathane Tubing) with specific sizes (0.160″ OD×0.091″ ID, 0.080″ OD×0.040″ ID & 0.025″ OD×0.012″ ID) (OD: outer diameter; ID: inner diameter) were purchased from Braintree Scientific Inc, USA. PU film was cast from PU solution prepared by dissolving catheters in DMF at 10% w/w concentration for 24 hours at 60° C. (C. Freij-Larsson, et al., J. Appl. Polym. Sci. 1993, 50, 345-352). The cationic monomers ((3-acrylamidopropyl) trimethylammonium chloride (AMPTMA), [2-(methacryloyloxy) ethyl] trimethylammonium chloride (MAETMA), and [2-(acryloyloxy) ethyl] trimethylammonium chloride (AETMA)) and anionic monomers (3-sulfopropyl methacrylate potassium (SPM), 3-sulfopropyl acrylate potassium (SPA), and 2-acrylamido-2-methylpropane sulfonate sodium (AMPA)), isopropyl alcohol (≥99.5%, ACS grade) were purchased from Sigma-Aldrich and used without further purification. Ammonium iron (ii) sulfate hexahydrate was purchased from Macklin, China. Ozone was generated by Azcozon RMU16-K3 ozone generator using air as oxygen source.

### General Methods

### Surface Analysis

The surface functional groups of modified catheters were characterized using Fourier-Transform Infrared Spectrometer (FT-IR) with ATR accessory at an incident angle of 90° (Nicolet 5700, Thermo Fisher Scientific, U.S.A), and X-ray photoelectron spectroscopy (XPS) (ESCALAB MK II spectrometer (VG Scientific Ltd, West Sussex, Britain) with a magnesium anode source). The molar ratio between N⁺(CH₃)₃/SO₃⁻ was calculated from the integration of relative peaks after plotting the spectra using OriginPro 2017. Contact angles were measured with a Goniometer (DSA 25, Kruss Scientific, Germany) and surface morphology was imaged by Field Emission Scanning Electron Microscopy (JEOL JSM-6701F, Japan). Height images were obtained with Atomic Force Microscopy (Dimension Icon, Bruker, USA) using tapping mode. The images and data were retrieved from Nanoscope Analysis software. Surface zeta potential was measured at pH 7 using a VisioLab Electro Kinetic Analyzer (Anton Paar, Germany). Viscosity of the polymer solution was measured using rheometer (UA-2000, TA instrument, New Castle, Del.) with a 1 mm gap for ramping on the 20 mm steel plate.

### Example 1

### Ozone Treatment of Catheters

A polyurethane (PU) catheter was first cut into 5 mm pieces and cleaned with methanol followed by deionized water. Air was used as input for ozone generation and the output ozone was purified by passing it through a 15 cm column filled with sodium hydroxide pellets. Peroxide groups were introduced on PU catheter surface by ozone treatment for 30 minutes at 15 L/min in a 100 mL enclosed vessel. Then, the ozone treated catheters were put under vacuum treatment (<10 Pa) for 1 hour to remove the oxygen/ozone which had diffused into the PU. For PU film, the film was cut into 1 cm*1 cm pieces prior to washing.

A 30 cm PU catheter was cleaned and activated using the same method in a 250 mL enclosed vessel.

### Coating

The procedures were illustrated using the 5 mm catheters pieces prepared above. 16 mmol each of cationic monomer AMPTMA and anionic monomer SPM was dissolved in 20 mL water and isopropanol (volume ratio 1:1) in a Schlenk tube to provide a monomer solution. The monomer solution was purged with Argon for 30 mins. Ozone treated 5 mm PU catheters were then placed into the monomer solution containing both monomers and the solution was Argon purged for 5 further minutes. 10 mg Ammonium iron (II) sulfate hexahydrate was then added to the solution. The polymerization reaction was aged for 24 hours at ambient temperature. The coated PU catheters were washed in deionized water to remove unreacted monomers and unattached homo- and co-polymers formed.

For 30 cm catheter coatings, a three-neck round bottom flask was used instead of a Schlenk tube with 100 mL of mixed solvent in total. The amount of monomers and iron (II) salt added were at the same concentrations as those for the small catheter piece coating. The catheter was connected to a peristatic pump with external tubing and fittings. The flow rate was set to 10 mL/min.

The procedures described above were used to manufacture the following polymers.
1. poly(AMPTMA-ran-SPM)—coating #1a
2. poly(AMPTMA-ran-SPA)—coating #2a
3. poly(AMPTMA-ran-AMPA)—coating #3a
4. poly(AETMA-ran-SPM)—coating #1b
5. poly(AETMA-ran-SPA)—coating #2b
6. poly(AETMA-ran-AMPA)—coating #3b
7. poly(MAETMA-ran-SPM)—coating #1c
8. poly(MAETMA-ran-SPA)—coating #2c
9. poly(MAETMA-ran-AMPA)—coating #3c
10. neat poly(AMPTMA)
11. neat poly(SPM)

Polymers 1-2, 4, 6, and 8-9 (i.e. Coatings #1a, #1b, #2a, #2c, #3b, and #3c) may form part of the invention, while polymers 3, 5, 7 (i.e. Coatings #1c, #2b, and #3a), 10 and 11 may be comparative samples.

### Surface Analysis

Fourier-transform Infrared-Attenuated total reflection (FTIR-ATR) and XPS were employed to evaluate the 9 prepared coatings. Both analyses indicated successful surface polymerization for all 9 coatings on the surface of the PU catheter.

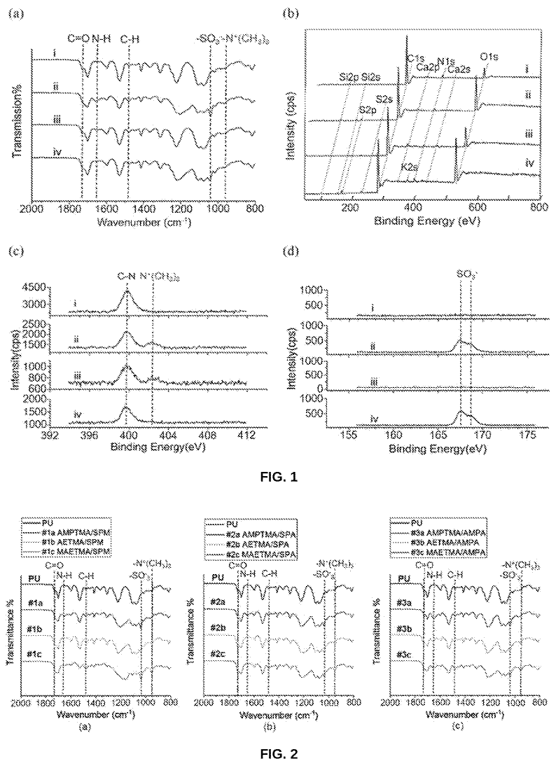

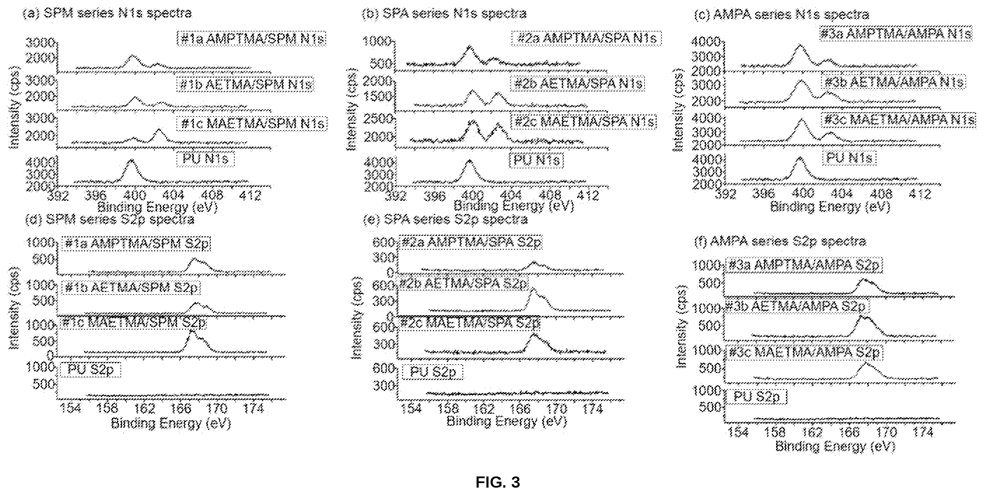

The poly(AMPTMA-ran-SPM) (coating #1a) is now described in detail. Compared with the unmodified PU catheter, after coating a PU catheter with the AMPTMA/SPM pair, neat AMPTMA or neat SPM (FIG. 1 (a) i, ii, iii & iv, respectively), the FTIR-ATR analysis shows a new peak at 1650 cm⁻¹ that is the characteristic peak of the secondary amine group of the acrylamide moiety of the AMPTMA monomer. The peak at 960 cm⁻¹, which is the characteristic peak of the quaternary amine group, also shows an increase in intensity after coating with AMPTMA (FIG. 1 (a) ii & iii). After coatings including SPM (FIG. 1 (a) ii & iv), a new peak at 1040 cm⁻¹, which is the characteristic peak of the sulfonate group, can be found. The polymerization also increased the intensities of signals from C—H (1485 cm⁻¹) and C═O (1735 cm⁻¹) groups on the surface (FIG. 1 (a) ii-iv), which are present in both AMPTMA and SPM. For other mixed-charge copolymer coatings (FIGS. 2(a) to 2(c)), the respective characterization peaks are also prominent, corroborating the successful surface polymerization of these monomers.

XPS was also to characterize the atoms present on the surfaces of the coated catheters. The unmodified PU catheter shows peaks of C1 s, O1s, N1s together with minor peaks of Si2p, Si2s and Ca2p, Ca2s (FIG. 1 (b) i). The peaks of Si and Ca elements are attributed to fumed silica and calcium carbonate which are commonly used inert additives to modify the mechanical property during PU fabrication process (J. Sepulcre-Guilabert, et al., Macromolecular Symposia 2001, 169185-190; and J. Sepulcre-Guilabert, et al., J. Adhes. Sci. Technol. 2001, 15, 187-203). The poly(AMPTMA-ran-SPM) coating (coating #1a) (FIG. 1 (b) ii) also shows peaks of C, O, N, Si and Ca elements just like those of unmodified PU catheter but also new S2p and S2s peaks, and these peaks are due to PU and also to the monomers of AMPTMA and SPM. The peak of K element in poly(SPM) coating (FIG. 1 (b) iv) is from K⁺ which is the counter ion in SPM salt, and does not show in the mixed-charge copolymer coating #1a (FIG. 1 (b) ii). The high resolution N1s peaks of coating #1a and the poly(AMPTMA) coating (FIG. 1 (c) ii & iii) at 422.5 eV and 399.8 eV are due respectively to —N⁺(CH₃)₃ of the quaternary ammonium group and C—N of the acrylamide of AMPTMA, corroborating the successful incorporation of AMPTMA into the coating. The poly(SPM) coating (FIG. 1 (c) iv) only shows one N1s peak at 399.8 eV due to C—N in PU. In coating #1a and poly(SPM) (FIG. 1 (d) ii & iv), the 164.1 eV peak is attributed to the S2p orbital in the sulfonate group; the poly(AMPTMA) (FIG. 1 (b) iii) does not have the S element. The other coatings also show similar XPS peaks corroborating the successful polymerization of other cationic/anionic pairs (FIGS. 3(a) to 3(f)).

The XPS atomic concentrations for PU, PU/AMPTMA, PU/SPM and PU/AMPTMA-ran-SPM were also obtained and are tabulated below in Table 1. The mixed-charge copolymer coating #1a (FIG. 1 (b)ii) has neither the Cl2p nor the K2s peak because the mobile counterions have been replaced by the charged monomers (Table 1).

TABLE 1

| | XPS atomic concentration % | | | | | | | |
|---|---|---|---|---|---|---|---|---|
| | C | N | O | S | Si | Ca | K | Cl |
| (i) Unmodified PU | 88.10 | 2.48 | 7.51 | — | 1.32 | 0.59 | — | — |
| (ii) Poly(SPM-ran-AMPTMA) | 71.92 | 2.99 | 19.33 | 2.42 | 3.13 | 0.21 | — | — |
| (iii) Poly(AMPTMA) | 83.16 | 2.13 | 13.00 | — | 0.19 | 0.98 | — | 0.54 |
| (iv) Poly(SPM) | 68.10 | 2.61 | 22.89 | 2.51 | 2.06 | — | 1.83 | — |

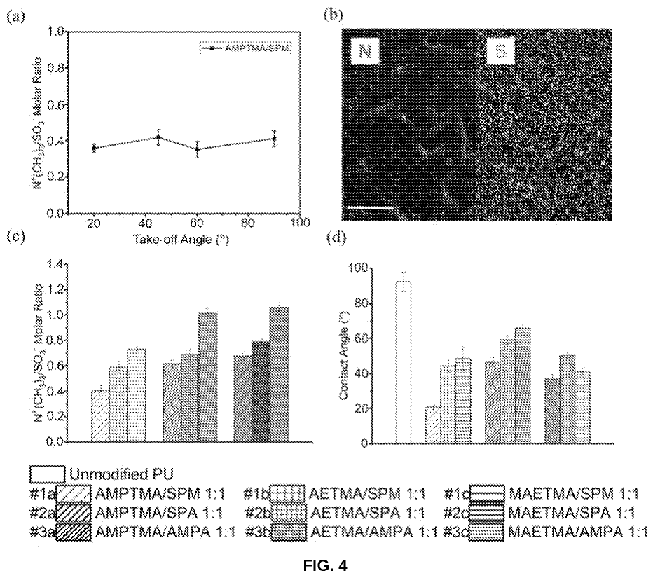

For poly(AMPTMA-ran-SPM) (coating #1a), XPS spectra were also measured at 4 different take-off angles from 20° to 90° (FIG. 4 (a)) to vary the depth of measurement penetration, and the N⁺(CH₃)₃/SO₃⁻ molar ratio was found to be almost constant at 0.4 and independent of the different X-ray penetration depths employed, indicating that the composition is almost constant through the measured thickness. Energy Dispersive X-ray (EDX) mapping also shows the enriched sulfur element (S) compared with nitrogen element (N) in coating #1a (FIG. 4 (b)).

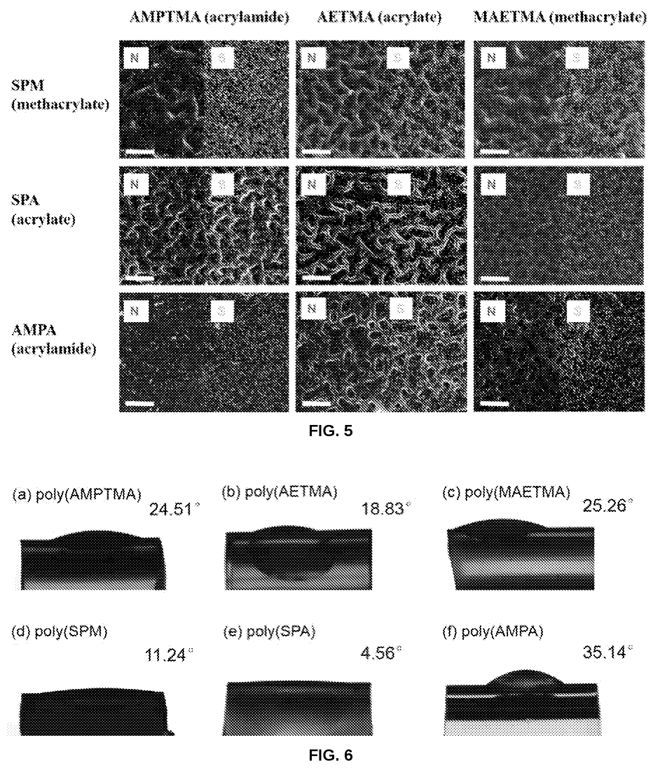

XPS at 90° take-off angle was also used to measure the molar ratio of N⁺(CH₃)₃ to SO₃⁻ groups of the other mixed-charge copolymer coatings (FIG. 4 (c)). Although the feed molar ratio was 1:1 for all combinations, the resulting copolymer coatings usually have N⁺(CH₃)₃/SO₃⁻ ratios that are smaller than 1.0. For the fixed anionic monomer SPM with different cationic monomers AMPTMA, AETMA and MAETMA, i.e. for coatings #1a, #1b and #1c, the N⁺(CH₃)₃/SO₃⁻ ratios are 0.41, 0.59 and 0.73 (i.e. all less 1.0) respectively. Similarly, for the other anionic monomer SPA, with cationic monomers AMPTMA, AETMA and MAETMA (for coatings #2a, #2b and #2c), the N⁺(CH₃)₃/SO₃⁻ ratios are 0.62, 0.69, and 1.01 respectively. The same trend occurs for the anionic monomer AMPA but with the highest N⁺(CH₃)₃/SO₃⁻ ratios: 0.68, 0.79 and 1.06 for coatings #3a, #3b and #3c respectively. EDX mapping images on other mixed-charge copolymer coatings also show the dominance of sulfur (S) element over nitrogen (N) element in the intensity of signals detected (FIG. 5).

Further, the hydrophilicity (and contact angles) of the mixed-charge surface coatings are also dependent on which cationic/anionic pair is used (FIG. 4 (d)). For all three sets of formulations with different anionic monomers, i.e. SPM, SPA or AMPA, the pair with AMPTMA (i.e. #1a, #2a and #3a) always had the lowest contact angle than those with other cationic monomers (#1a versus #1b, #1c; #2a versus #2b, #2c; and #3a versus #3b, #3c) as the sulfonate content in AMPTMA-containing coatings (#1a, #2a, #3a) is the highest (FIG. 4 (c) & (d)). AMPTMA/SPM coating (#1a) is the most hydrophilic among all 9 formulations. MAETMA/AMPA (#3c) deviates from the general trend, with relatively lower contact angle but high N⁺(CH₃)₃/SO₃⁻ ratio, as the poly(AMPA) coating is the least hydrophilic amongst the three cationic homopolymer coatings (FIG. 6).

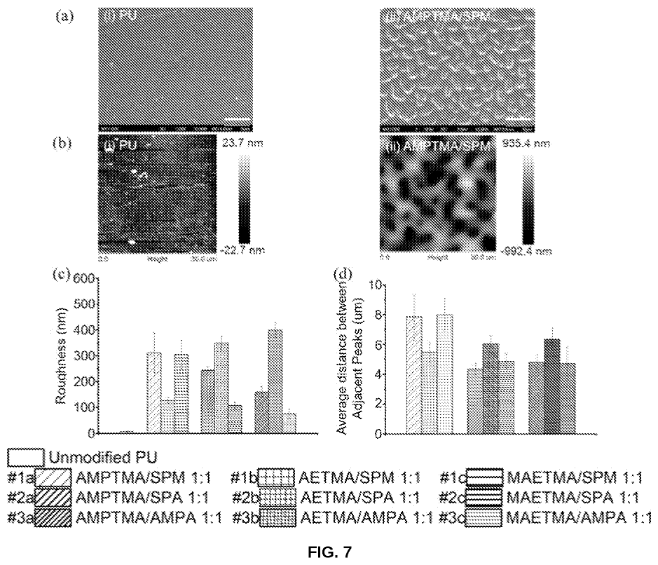

The control (unmodified PU catheter) and poly(AMPTMA-ran-SPM) coating (coating #1a) morphologies were characterized by FESEM, Atomic force microscopy (AFM) and EDX mappings. Both FESEM and AFM height imaging (FIG. 7 (a) & (b)) show that the surface of the unmodified PU catheter is almost featureless with a root-mean-square roughness (Rq) of 6.97±2.51 nm (FIG. 7 (c)). On the other hand, coating #1a shows a dimple structure with a Rq of 311.33±78.32 nm (FIG. 7 (c), #1a). The root-mean-square distance between adjacent peaks of coating #1a is 7.84±1.56 μm (FIG. 7 (d), #1a) as measured by AFM section mode. We also analyzed the morphology of other mixed-charge copolymer coatings using AFM; all 9 mixed-charge copolymer coatings have similar low-frequency roughness which are in the range of 75.43±18.28 nm to 399.18±28.34 nm (FIG. 7 (c)). The average distance between adjacent peaks are in the range of 4.35±0.39 μm to 7.98±1.10 μm (FIG. 3 (d)). Dimple structures can also be observed on the other 8 mixed-charge copolymer coatings in the EDX mapping images (FIG. 5).

The zeta potential of all the mixed-charge copolymer coatings was measured and is presented in FIG. 8. Generally, the zeta potential has a small net negative charge (−2 mV to −5 mV). The slight negative surface charge state is important for the coatings to be bacteria-resistant as both Gram-positive and Gram-negative bacteria adhere preferentially to positively charged surfaces (B. Gottenbos, et al., J. Antimicrob. Chemother. 2001, 48, 7-13).

### Comparative Example 1

### Polymerization Kinetics Measurements

Individual monomer solutions (each containing only one of AMPTMA, AMPA, AETMA, SPA, MAETMA and SPM) were prepared at 1.6 mol/L in 20 mL water and isopropanol (volume ratio 1:1) mixture. Each monomer solution was purged for 30 mins in a Schlenk tube using Argon. 10 μL of 30% H₂O₂ was added to the monomer solution as the initiator and the solution was purged for 5 further mins. 10 mg Ammonium iron (ii) sulfate hexahydrate was then added to the solution to initiate the Fenton reaction. At predetermined time points, 50 μL of the reaction mixture was removed. The polymer in these samples was precipitated in 1 mL acetonitrile containing 1% hydroquinone and centrifuged at 7,000 rpm for 5 minutes. The supernatant was removed and the precipitates were dissolved in deuterated water for NMR analysis at 300 MHz in Bruker Avance DPX 300 instrument.

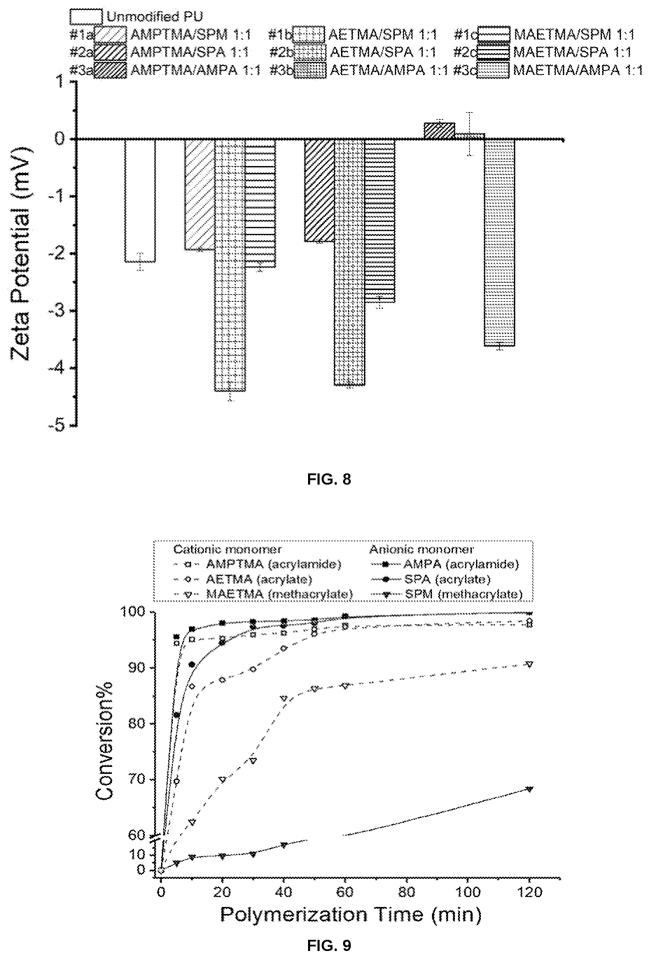

The polymerization kinetics of the individual monomers were obtained by analysis of the liquid phase composition with NMR (FIG. 9). Without wishing to be bound by theory, the ratio of N⁺(CH₃)₃ to SO₃⁻ groups on the solid catheter coating surface for each of the coatings #1a-c, #2a-c, and #3a-c seems to be correlated to the solution phase polymerization kinetics. For the monomer which polymerized faster in the solution phase, its incorporation into the coating growth was observed to be slower in turn. For example, the anionic monomer SPM has the slowest solution polymerization rate amongst the anionic monomers investigated (i.e. SPM, SPA and AMPA) (FIG. 9), but the SPM-containing coatings (#1a, #1b, #1c) (FIG. 4 (c)) generally have lower N⁺(CH₃)₃/SO₃⁻ ratio (i.e. higher SO₃⁻/N⁺(CH₃)₃ than the other formulations containing other anionic monomers (SPA and AMPA)), while keeping the cationic monomer the same. For example, coating #1a has lower N⁺(CH₃)₃/SO₃⁻ (i.e. higher SO₃⁻/N⁺(CH₃)₃ ratio) than coatings #1b and #1c. Further, comparing coating #1a, #1b and #1c with the same anionic SPM, the coating with cationic monomer AMPTMA has the lowest N⁺(CH₃)₃/SO₃⁻ ratio (0.41), compared to the other coatings with cationic monomers AETMA and MAETMA, because AMPTMA has the fastest solution polymerization rate. With anionic SPM, pairing with cationic methacrylate (MAETMA, coating #1c) produced the highest N⁺(CH₃)₃/SO₃⁻ ratio (0.73) (FIG. 4 (c)), as the polymerization rate in solution of MAETMA is the slowest among the cationic monomers (FIG. 9). Among the 9 formulations, poly(AMPTMA-ran-SPM) coating (coating #1a) has the lowest N⁺(CH₃)₃/SO₃⁻ ratio as the cationic monomer AMPTMA used has the fastest solution polymerization rate among the three cationic monomers while the anionic monomer SPM used has the slowest solution polymerization rate among the three anionic monomers.

### Example 2

### In Vitro Antibiofilm Efficacy

Unmodified and modified catheters were soaked in 10 mM phosphate buffered saline (PBS) for at least 1 day and sterilized under UV lamp (30W, G30T8, Sankyo Denki) for 30 mins prior to testing. To quantify the amount of biofilm grown on the mixed-charge copolymer coated catheter surface in vitro, Methicillin-resistant Staphylococcus aureus (MRSA BAA38), Methicillin-resistant Staphylococcus epidermidis (MRSE 35984), Vancomycin-resistant Enterococcus faecalis (VRE V583), Pseudomonas aeruginosa PAO1, Escherichia coli UT189 and Acinetobacter baumannii AB-1 were used as targets. All of the mixed-charge copolymer coating formulations were challenged with these clinically important Gram-positive and Gram-negative bacteria using an anti-biofilm protocol (C. D. Blanco, et al., ACS Appl. Mater. Interfaces 2014, 6, 11385-11393) as described below.

Overnight culture of bacteria was prepared in Mueller Hinton Broth (MHB) medium from bacteria colonies grown on Luria-Bertani Agar (LB Agar) plates and diluted in 1:100 volume ratio for subculture. Bacteria suspension was prepared at 10⁷ CFU/mL concentration in Tryptic Soy Broth (TSB) medium. Then the unmodified and modified catheters were incubated with the prepared bacteria suspension for 24 hours at 37° C. After incubation, catheters were carefully removed and rinsed 3 times in PBS. After ultra-sonication in ice bath and vortexing, the obtained bacteria suspensions were serial diluted and plated on LB Agar for CFU counting. Log₁₀ reduction was calculated using Equation (1):

$$Number of {log}_{10} reduction = {log}_{10} [ \frac{{CFU}_{control}}{{CFU}_{sample}} ] \qquad ( 1 )$$

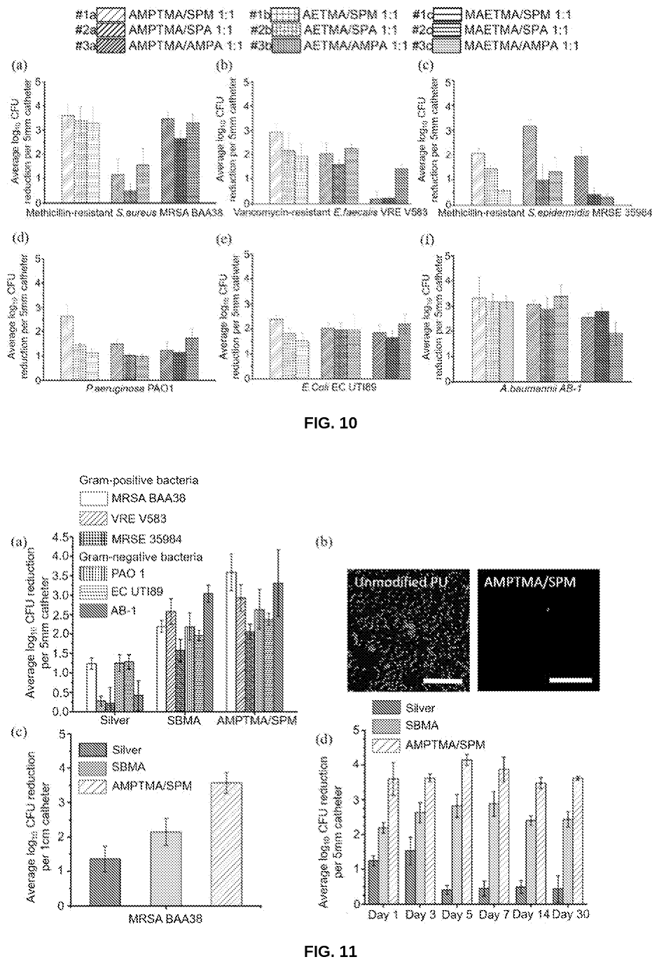

The results are shown in FIG. 10 and Table 2. MRSA is an important pathogen to combat for CRIs, but the coatings from the SPA series (#2a, #2b, #2c) were ineffective towards this bacteria (FIG. 10 (a)). Also, the coatings from the AMPA series (#3a, #3b, #3c) were ineffective towards E. faecalis, leaving the coatings from the SPM series (#1a, #1b, #1c) for further studies (FIG. 10 (b)). Of the coatings from the SPM series, only coating #1a (AMPTMA/SPM) was effective (with >2 log₁₀ reduction) against S. epidermidis and P. aeruginosa (FIG. 10 (c) & (d)). For E. coli and A. baumanii, the 9 formulations including coating #1a, all appeared fairly effective (with >2 log₁₀ reductions) (FIG. 10 (e) & (f)). So coating #1a (poly(AMPTMA-ran-SPM)) appeared to be the most broad-spectrum effective.

TABLE 2
24 hours' static biofilm assay results with average log₁₀ CFU reduction against 6 species of bacteria.

| | Anionic monomer | | | | | | | | |
|---|---|---|---|---|---|---|---|---|---|
| Cationic | SPM | | | SPA | | | AMPA | | |
| monomer | AMPTMA | AETMA | MAETMA | AMPTMA | AETMA | MAETMA | AMPTMA | AETMA | MAETMA |
| MRSA BAA38 | 3.59 | 3.38 | 3.29 | 1.15 | 0.49 | 1.55 | 3.47 | 2.65 | 3.3 |
| MRSE 35984 | 2.05 | 1.45 | 0.56 | 3.17 | 0.98 | 1.31 | 1.94 | 0.41 | 0.3 |
| VRE V583 | 2.93 | 2.17 | 1.92 | 2.03 | 1.58 | 2.26 | 0.2 | 0.22 | 1.42 |
| EC UT189 | 2.38 | 1.8 | 1.52 | 2.03 | 1.94 | 1.94 | 1.83 | 1.65 | 2.2 |
| PAO1 | 2.63 | 1.46 | 1.12 | 1.49 | 1.03 | 0.98 | 1.24 | 1.14 | 1.74 |
| AB-1 | 3.31 | 3.19 | 3.17 | 3.07 | 2.87 | 3.39 | 2.55 | 2.78 | 1.91 |

### Comparative Example 2

Poly(SBMA) coated PU catheter was prepared from 16 mmol of SBMA monomer based on the protocol described in Example 1.

### Example 3

### Comparison Using In Vitro Antibiofilm Efficacy Test

Poly(SBMA) (comparative Example 2), the commercially available Palindrome™ silver hydrogel catheter and coating #1a (poly(AMPTMA-ran-SPM)) were subjected to the in vitro antibiofilm efficacy test set out in Example 2 using the same six bacteria.

In FIG. 11 (a), poly(AMPTMA-ran-SPM) coating shows broad spectrum anti-biofilm activities against the 6 bacteria species with log₁₀ reductions ranging from 2.05 to 3.59 (99.11%-99.97%). The anti-biofilm efficacy of poly(AMPTMA-ran-SPM) coating against each tested pathogen was generally better than the commercial silver catheter by 1.10-2.90 log₁₀ reductions, and poly(SBMA) coating by 0.27-1.39 log₁₀ reductions respectively.

Comparison Using Intraluminal Dynamic Antibiofilm Test with MRSA BAA38

Protocol for this test was adopted from literature (R. S. Smith, et al., Sci. Transl. Med. 2012, 4, 153ra132). Unmodified and modified 30 cm catheters were soaked in 10 mM PBS for at least 1 day and sterilized under UV lamp for 30 mins prior to testing. Overnight culture of MRSA BAA38 was prepared in MHB medium and diluted in 1:100 volume ratio for subculture. Bacteria suspension was prepared at 10⁷ CFU/mL concentration in PBS. The catheter was pre-incubated with 50% human serum for 30 mins, followed by 2 hours' incubation with bacteria suspension at 37° C. The pre-incubated catheter was put into 10% Brain Heart Infusion (BHI) medium. The system was connected to a peristatic pump which circulated the nutrient medium at 2 mL/min through the catheter for 24 hours at 37° C. After incubation, the catheter was rinsed with PBS for 10 mins and cut into 5 cm segments. Each segment was cut into 5 pieces with 1 cm length and the two pieces from each end were discarded. Then, each piece was put into 1.5 mL PBS and sonicated for 10 mins. The suspension was then vortexed and diluted for plating on LB Agar plates. CFU was counted after 24 hours incubation at 37° C. and the log₁₀ reduction was calculated using Equation (1).

### Live & Dead Confocal Microscope Imaging of MRSA BAA38 Biofilm Grown on Catheter Material Film

Unmodified and modified PU films were incubated with MRSA BAA38 as described above. For imaging, the films were gently rinsed 3 times with PBS and stained with SYTO 9 and propidium iodide for 15 minutes. Red and green fluorescence images were obtained using a Zeiss LSM780 confocal laser scanning microscope (Carl Zeiss, Jena, Germany) with a 63× oil immersion objective lens at 488 nm and 561 nm excitation wavelengths.

### Discussion

Using MRSA BAA38, a bacteria strain isolated from human blood (S. Monecke, et al., PloS one 2011, 6, e17936), it was found that poly(AMPTMA-ran-SPM) coating has a good anti-biofilm activity of 3.59 log₁₀ reduction (˜99.97%). Confocal microscopy images (FIG. 11 (b)) of Live/Dead fluorescently stained biofilm also show decreased numbers of MRSA BAA38 bacteria on poly(AMPTMA-ran-SPM) coating compared with the unmodified PU catheter. On the other hand, against MRSA BAA38, the poly(SBMA) coating produced 2.20 log₁₀ reduction (˜99.37%), while the commercial silver catheter produced 1.24 log₁₀ reduction (˜94.25%) (FIG. 11 (a)). The dynamic antibiofilm efficacy of the poly(AMPTMA-ran-SPM) coating was also tested by making the coating on a 30 cm PU catheter. The poly(AMPTMA-ran-SPM) coated catheter inhibits biofilm formation with 3.57 log₁₀ reduction (˜99.97%) under dynamic bacterial flow for 24 hours compared with unmodified PU catheter (FIG. 11 (c)). The poly(SBMA) coating and commercial silver catheter produced 2.15 log₁₀ reduction (˜99.29%) and 1.36 log₁₀ reduction (˜95.63%) respectively.

Using MRSA BAA38 as the target strain, the poly(AMPTMA-ran-SPM) coated PU catheter was also tested for long-term antibiofilm efficacy with an incubation period of up to 30 days. Unlike the commercial silver catheter, which became less effective by the 5th day, both poly(AMPTMA-ran-SPM) coated catheter and poly(SBMA) coated catheter maintained their antibiofilm efficacy for the full 30 days of the experiment (FIG. 11 (d)). The log₁₀ reduction of poly(AMPTMA-ran-SPM) coating and poly(SBMA) coating against MRSA after 30 days is 3.62 (99.98%) and 2.44 (99.64), consistent with the results of the 24 hours' assay (FIG. 11 (a)). This consistency between short-term and long-term results indicates the potential of poly(AMPTMA-ran-SPM) coating in long-term catheterization applications.

### Example 4

Solution Viscosity of Poly(AMPTMA-Ran-SPM) Versus SBMA after Polymerization

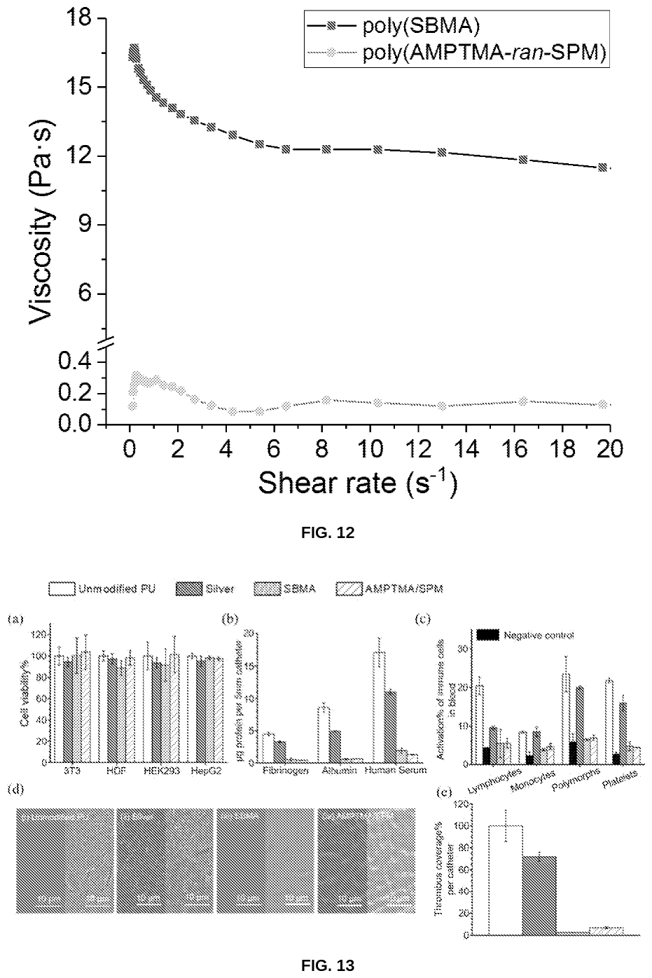

A solution with high viscosity can make it difficult to remove coated catheters from the reaction vessel and can also cause problems for the subsequent washing steps after polymerization (e.g. see T. Richey, et al., Biomaterials 2000, 21, 1057-1065). To quantify the solution viscosity of poly(AMPTMA-ran-SPM), polymer solutions (made according to the protocol of Example 1) were taken out after 24 hours' polymerization and measured using a rheometer and compared with poly(SBMA) as made in Comparative Example 2 (FIG. 12). Five viscosity values were retrieved from the plateau region in each curve, and summarized in Table 3. It shows that the poly(SBMA) solution is ˜86.77 fold more viscous than poly(AMPTMA-ran-SPM) solution; the average viscosities of poly(SBMA) and poly(AMPTMA-ran-SPM) are 12.01 Pa-s, and 0.14 Pa-s respectively. The higher solution viscosity of poly(SBMA) is due to its poor solubility (J. Yu, et al., Polym. J, 2017, 49, 767; Y. Fan, et al., Polymers 2019, 11, 192; and N Wang, et al., Polym. Chem, 2018, 9, 5257-5261). However, to fabricate a coating on the inner surface of a thin and long catheter tubing, it is necessary to infuse the monomer solutions into the lumen for polymerization. A solution with a viscosity lower than 5 Pas is preferred in this case (A. Von Holst, et al. (Google Patents, U.S. Pat. No. 8,287,940B2, 2012); S. A. Herweck, et al. (Google Patents, U.S. Pat. No. 7,947,015B2, 2011); and M. Hoffmann, et al. (Google Patents, US20120316496A1, 2012)). The high viscosity of poly(SBMA) (˜12.01 Pa·s) may cause the unbounded polymer to be retained in the catheter lumen and makes it difficult to be fully washed away. The unwashed polymer residue on the coating can bring about flaws and may decrease the coating performance. Hence, a less viscous polymer solution, such as the poly(AMPTMA-ran-SPM) solution, can prevent this issue at the laboratory level synthesis and for larger scale production in the future.

TABLE 3
Viscosity of polymer solution after 24 hours' polymerization.

| | Viscosity (Pa · s) | | |
|---|---|---|---|
| Shear | | poly | Viscosity _{poly(SBMA)}/ |
| rate | poly | (AMPTMA- | Viscosity |
| (s⁻¹) | (SBMA) | ran-SPM) | _{poly(AMPTMA-ran-SPM)} |
| 8.19 | 12.30 | 0.16 | 78.34 |
| 10.32 | 12.28 | 0.14 | 87.84 |
| 12.99 | 12.15 | 0.12 | 102.53 |
| 16.37 | 11.84 | 0.15 | 79.20 |
| 19.69 | 11.50 | 0.13 | 90.20 |
| Average | 12.01 | 0.14 | 86.77 |

### Example 5

### In Vitro Cytotoxicity Assay

Unmodified and modified catheters were soaked in 10 mM PBS for at least 1 day and sterilized under UV lamp for 30 mins prior to testing. Each unmodified and modified catheter was soaked in 1 mL supplemented Dulbecco's Modified Eagle's Medium (DMEM with 10% FBS and 1% Penicillin-Streptomycin) for 72 hours at 37° C. to extract leachable molecules. 200 μL 104 cells/well 3T3 cells suspension in supplemented DMEM were seeded in 96-well cell culture plate and placed in a CO₂ incubator for 24 hours at 37° C. After incubation, the medium in the culture plate was carefully removed by pipettes, and replaced with an equal volume of the catheter extraction medium and the cultures were incubated for another 24 hours at 37° C. For control wells, only supplemented DMEM was added for cell incubation. Subsequently, all of the medium was carefully removed and replaced with 100 μL of 3-(4,5-dimethylthiazol-2-yl)-2,5-diphenyltetrazolium bromide (MTT tetrazolium dye) dissolved in pure DMEM at 1 mg/mL concentration. Then the 96-well plate was incubated for 4 hours. 100 μL dimethyl sulfoxide (DMSO) was added to each well after discarding the MTT-DMEM solution. Absorbance values (OD) were measured at 570 nm using TECAN plate reader. Cell viability was calculated using Equation (2):

$$\% Cell viability = \frac{Average absorbance from treated cells \times 100 \%}{Average absorbance from cells in the control wells} \qquad ( 2 )$$

To test the biocompatibility of poly(AMPTMA-ran-SPM) coating with mammalian cells, the poly(AMPTMA-ran-SPM) coated catheter was placed in cell culture medium and incubated for 72 hours at 37° C. as described above to provide an extract that was subjected to an MTT assay. The MTT assay for toxicity of the extract was conducted afterwards with 4 types of commonly used mammalian cells as described above. The results show that the poly(AMPTMA-ran-SPM) coating is non-toxic to mammalian cells (FIG. 13 (a)).

### Example 6

### Protein Antifouling Assay

Unmodified and modified PU catheters (unmodified PU catheter, commercial silver catheter as mentioned above, poly(AMPTMA-ran-SPM) coated PU catheter, and poly(SBMA) coated PU catheter) were soaked in 10 mM PBS for at least 1 day and sterilized under UV lamp for 30 mins prior to testing. The catheters were immersed in 10 mg/mL protein solution prepared with 10 mM PBS and incubated at 37° C. for 24 hours on a shaker at 110 rpm. After incubation, the catheters were removed and washed 3 times with PBS. The washed catheters were then put into 1 mL of 1% SDS solution with shaking at 110 rpm for 2 hours and were then sonicated for 10 mins. The supernatants were then examined using BCA protein assay kit according to the supplier's protocol. OD values at 562 nm wavelength were measured and protein concentrations were calculated according to the calibration curve provided in the kit.

To assess the antifouling performance of poly(AMPTMA-ran-SPM) in a complex environment, the coating was challenged with three types of proteins, i.e. human serum, fibrinogen and bovine serum albumin, over a 24-hour incubation period. A colorimetric method was employed to quantify the protein adsorbed on the catheter surface (S. Braune, et al., Clin. Hemorheol. Microcirc. 2015, 61, 225-236). After 24 hours' incubation, poly(AMPTMA-ran-SPM) coating showed much lower protein adsorption compared with unmodified PU catheter and commercial silver catheter, and similar performance to poly(SBMA) coating (FIG. 13 (b)).

### Example 7

### Quantification of Activated Cells in Rabbit Blood by Immunostaining and Flow Cytometry

Unmodified and modified catheters (unmodified PU catheter, commercial silver catheter as mentioned above, poly(AMPTMA-ran-SPM) coated PU catheter, and poly(SBMA) coated PU catheter) were soaked in 10 mM PBS for at least 1 day and sterilized under UV lamp for 30 mins prior to testing. Then each catheter segment was incubated with 100 μL of freshly-drawn rabbit blood for 30 minutes. After catheter removal, the blood cells were then fixed with 100 μL 4% paraformaldehyde for 10 minutes at room temperature in the glass vials. The excess paraformaldehyde was then removed by centrifugation, and the blood cells were carefully washed twice with PBS. Specific antibodies were dissolved in PBS at 1% (v/v) concentration and incubated with blood cells for 1 hour (Lymphocytes: CD3-APC and CD25-FTIC; monocytes: CD14-APC, CD11b-FTIC; platelets: CD41-FTIC, CD62p-APC; Polymorphs: CD66b-APC, CD11-FTIC). The labelled blood cells were then washed twice with PBS. Cell counts were performed using Attune N×T Acoustic Focusing Cytometer. Blood without any treatment was used as negative control.

Blood immunological assay was performed by incubating poly(AMPTMA-ran-SPM) coated catheter in freshly drawn rabbit blood for 2 hours at 37° C. Fluorophore-tagged primary anti-bodies were used to label activated cells, which were counted by flow cytometry. The results (FIG. 13 (c)) show that, compared with unmodified PU catheter and the commercial silver catheter, both the poly(AMPTMA-ran-SPM) coating and the poly(SBMA) coating elicit lower immune response in monocytes, lymphocytes, polymorphs and platelets.

### Example 8

Thrombus Formation Evaluation with FESEM

Unmodified and modified catheters were incubated with 1 mL of freshly-drawn rabbit blood for 2 hours, and gently washed 3 times with PBS. Then the catheters were fixed with 4% paraformaldehyde for 8 hours at 4° C. After dehydration in 25% ethanol, 50% ethanol, 75% ethanol and 100% ethanol, the fixed samples were dried in air at ambient temperature before Field Emission Scanning Electron Microscopy (FESEM) imaging.

### Acid Phosphatase Assay

The protocol was adapted as previously described (S. Braune, et al., Clin. Hemorheol. Microcirc. 2015, 61, 225-236). Unmodified and modified catheters were incubated with 1 mL of freshly-drawn rabbit blood for 2 hours, and gently washed 3 times with PBS. Then each catheter was immersed in 100 μL of 0.1 mol/L citrate buffer (pH 5.4) containing 5 mM p-nitrophenylphosphate and 1% (v/v) Triton X-100. After 1 hour of incubation at 37° C., the reaction was stopped by adding 70 μL of 2 mol/L NaOH stop solution. The final solution was transferred into a 96-well plate and the absorbance was read at a wavelength of 405 nm. The percentage of platelet proxy thrombus coverage was calculated using Equation (3):

$$\% Thrombus converage = \frac{Average absorbance from sample catheter \times 100 \%}{Average absorbance from PU control catheter} \qquad ( 3 )$$

### Discussion

To qualitatively evaluate thrombus formation on the catheter surface, the catheters were incubated with rabbit blood for 2 hours, then they were fixed with 4% paraformaldehyde for FESEM imaging (FIG. 13 (d)). Both PU catheter and commercial silver catheter surfaces were covered with thrombi after incubation. In contrast, no obvious thrombus was formed on either poly(AMPTMA-ran-SPM) coating or poly(SBMA) coating. As thrombus formation is correlated with platelet aggregation (W. S. Nesbitt, et al., Nat. Med. 2009, 15, 665) we also performed the acid phosphatase assay to quantify platelets on the catheter surface after incubation. The platelets quantified by this colorimetric method were used to represent the amount of thrombus coverage on the catheter surface (FIG. 13 (e)). By this measure, poly(AMPTMA-ran-SPM) coated catheter and poly(SBMA) coated catheter reduced thrombus coverage by over 90% compared to the unmodified PU catheter, while the commercial silver catheter reduced thrombus coverage by 28%. In addition to low immune response, poly(AMPTMA-ran-SPM) coating can also reduce thrombosis on the catheter surface, which is an important consideration for possible application in devices which contact blood (B. A. Butruk-Raszeja, et al., Colloids Surf. B. Biointerfaces 2015, 130, 192-198).

### Example 9

### In Vivo Antibiofilm Assay Using Mouse Urinary Model

Animals were implanted with catheters and infected with the indicated bacterial strain as previously described (C.-S. Hung, et al., Nat. Protoc. 2009, 4, 1230). Briefly, 7 to 8 weeks' old female mice were anesthetized by inhalation of isoflurane and transurethrally implanted with coated and uncoated PU catheter tubing (5 mm in length, 0.025″ OD×0.012″ ID). Immediately after implantation, 50 μL of 10⁷ CFU/mL bacteria suspension in PBS buffer was introduced into the bladder lumen by transurethral inoculation. At the 24-hour time point, animals were euthanized with CO₂ followed by cervical dislocation. Bladders and kidneys were aseptically harvested. Afterwards, the PU catheters were retrieved from the bladder, placed in PBS, ultra-sonicated for 10 mins, and then vortexed at maximum speed for 5 mins. Samples were serially diluted and plated on LB agar plates. CFU were enumerated after 24 hrs of incubation at 37° C.

### In Vivo Antibiofilm Assay Using Mouse Subcutaneous Model

Overnight culture of bacteria was prepared in Mueller Hinton Broth (MHB) medium and diluted in 1:100 volume ratio for subculture. Bacteria suspension was prepared at 10⁷ CFU/mL concentration in 1% TSB medium in PBS. Both unmodified and modified catheters (5 mm in length, 0.080″ OD×0.040″ ID) were incubated with bacteria suspension at 37° C. for 2 hours. 7 to 8 weeks' old female Balb/c mice were anesthetized by injection of ketamine/xylazine and shaved. 5 mm diameter incisions were made on the back of the mice. Each catheter was removed from the bacteria suspension, washed twice with PBS, and then inserted into the subcutaneous pocket of the incision created on the back of mice. The incision was then sealed with 3M Tegaderm™. At the 24-hour time point, all mice were euthanized with CO₂. The implanted catheters were then removed and put into 1 mL PBS. The samples were then ultra-sonicated for 10 mins to remove the adhered bacteria, and serially diluted for plating on LB agar plates. CFU were enumerated after 24 hours of incubation at 37° C. The logo reduction was calculated using Equation (1).

### Discussion

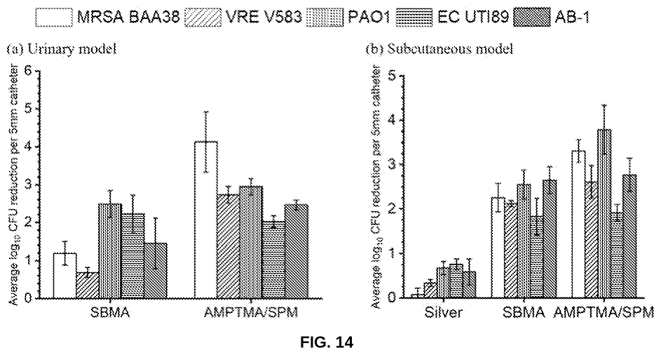

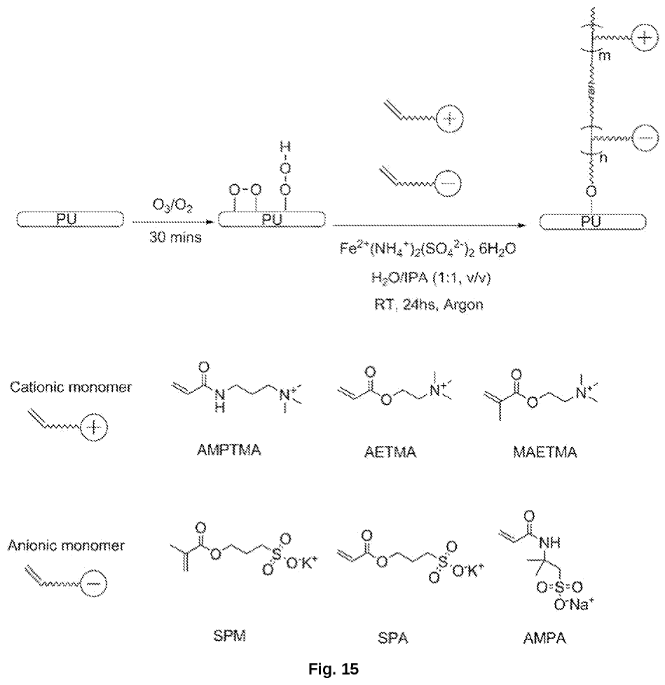

Urinary and blood-contacting catheters are widely used in healthcare. To evaluate the in vivo anti-biofilm activity, the poly(AMPTMA-ran-SPM) coated catheter was applied in both the urinary tract infection model (C.-S. Hung, et al., Nat. Protoc. 2009, 4, 1230) and subcutaneous wound infection model (J. L. Kadurugamuwa, et al., Infect. Immun. 2003, 71, 882-890) against relevant bacteria. For the urinary tract infection model, the coated catheter and bacteria suspension were pushed into the bladder through the urinary tract and incubated there for 24 hours. For subcutaneous wound infection model, the coated catheter was infected with bacteria before implantation, then sealed into a subcutaneous pocket on the mouse back for 24 hours. In both models, the biofilm grown on the catheter was stripped off using ultra-sonication and vortexed for plating and colony counting. Poly(AMPTMA-ran-SPM) coating has comparable or better anti-biofilm efficiency than poly(SBMA) coating against 5 selected bacteria in both models (FIG. 14). Hence, the in vivo performance of poly(AMPTMA-ran-SPM) coating is consistent with its in vitro performance (FIG. 11 (a)).

1. A composite material suitable for inhibiting biofilm growth, the composite material comprising:
a substrate material; and
a random copolymeric material covalently bonded to a surface of the substrate material, wherein the random copolymeric material has a formula I:
[E]-[G] I
where:
E represents a repeating unit derived from a monomer J, where the monomer J has at least one functional group bearing a cationic charge; and
G represents a repeating unit derived from a monomer L, where the monomer L has at least one functional group bearing an anionic charge, wherein:
the monomers J and L belong to different, but compatible, monomer classes with differing polymerisation kinetics.

2. The composite material according to claim 1, wherein the monomer classes are acrylamide, alkylacrylamide, acrylate, alkylacrylate, and vinyl monomers, wherein the vinyl monomers are selected from vinyl benzyl monomers and vinyl benzene monomers.

3. The composite material according to claim 1, wherein the random copolymeric material is selected from:
(Ai) poly(cationic acrylamide-ran-anionic methacrylate);
(Aii) poly(cationic acrylamide-ran-anionic acrylate);
(Aiii) poly(cationic acrylamide-ran-anionic methacrylamide);
(Aiv) poly(cationic acrylamide-ran-anionic vinyl monomer)
(Av) poly(cationic methacrylate-ran-anionic acrylate);
(Avi) poly(cationic methacrylate-ran-anionic acrylamide);
(Avii) poly(cationic methacrylate-ran-anionic methacrylamide);
(Aiii) poly(cationic methacrylate-ran-anionic vinyl monomer)
(Aix) poly(cationic acrylate-ran-anionic methacrylate);
(Ax) poly(cationic acrylate-ran-anionic acrylamide);
(Axi) poly(cationic acrylate-ran-anionic methacrylamide);
(Axii) poly(cationic acrylate-ran-anionic vinyl monomer);
(Axiii) poly(cationic vinyl monomer-ran-anionic methacrylate);
(Axiv) poly(cationic vinyl monomer-ran-anionic acrylate);
(Av) poly(cationic vinyl monomer-ran-anionic acrylamide); and
(Avi) poly(cationic vinyl monomer-ran-anionic methacrylamide).

4. The composite material according to claim 1, wherein the at least one functional group bearing an anionic charge in monomer L is selected from one or more of the group consisting of sulfonic, carboxylate, and phosphate.

5. The composite material according to claim 4, wherein the at least one functional group in monomer L is a sulfonic functional group.

6. The composite material according to claim 1, wherein the at least one functional group bearing a cationic charge in monomer J is selected from one or more of the group consisting of phosphonium, imidazolium, and ammonium.

7. The composite material according to claim 6, wherein the at least one functional group in monomer J is an ammonium functional group.

8. The composite material according to claim 1, wherein monomer J is selected from (3-acrylamidopropyl) trimethylammonium chloride (AMPTMA), [2-(methacryloyloxy) ethyl] trimethylammonium chloride (MAETMA), [2-(acryloyloxy) ethyl] trimethylammonium chloride (AETMA), [3-(methacryloylamino)propyl] trimethylammonium chloride (MAPTAC), (3-acrylamidoethyl) methylimidazolium chloride (AMEMI), [2-(methacryloyloxy) ethyl] methylimidazolium chloride (MAEMI), [2-(acryloyloxy) ethyl] methylimidazolium chloride (AEMI), [3-(methacryloylamino)ethyl] methylimidazolium chloride (MAAEMI), (vinylbenzyl) trimethylphosphonium chloride (VBTMP), and (vinylbenzyl) trimethylammonium chloride (VBTMA).

9. The composite material according claim 1, wherein monomer L is selected from 3-sulfopropyl methacrylate potassium (SPM), 3-sulfopropyl acrylate potassium (SPA), 2-acrylamido-2-methylpropane sulfonate sodium (AMPA), 3-sulfopropyl methacrylamide potassium (SPMA), 2-carboxyethyl acrylate (CEA), 2-carboxyethyl methacrylate (CEM), 2-carboxylethyl acrylamide (CEAM), 2-carboxylethyl methacrylamide (CEMA), 4-vinylbenzoic acid (VBA), and sodium 4-vinylbenzenesulfonate (VBS).

10. The composite material according to claim 9, wherein monomer L is selected from 3-sulfopropyl methacrylate potassium (SPM), 3-sulfopropyl acrylate potassium (SPA), and 2-acrylamido-2-methylpropane sulfonate sodium (AMPA).

11. The composite material according to claim 1, wherein the random copolymeric material is selected from:
(ai) poly(AMPTMA-ran-SPM);
(aii) poly(AMPTMA-ran-SPA);
(aiii) poly(AETMA-ran-SPM);
(aiv) poly(AETMA-ran-AMPA);
(av) poly(MAETMA-ran-SPA); or
(avi) poly(MAETMA-ran-AMPA).

12. The composite material according to claim 11, wherein the random copolymeric material is poly(AMPTMA-ran-SPM).

13. The composite material according to claim 1, wherein the substrate material is a polyurethane.

14. The composite material according to claim 1, wherein the composite material has a water contact angle of less than 50°.

15. An article comprising a composite material as described in claim 1.

16. The article according to claim 15, wherein the article is a catheter.

17. A method of forming a composite material as described in claim 1, the method comprising the steps of:
(aa) providing a mixture comprising a substrate material, a solvent, a monomer J that has at least one functional group that bears a cationic charge, and a monomer L that has at least one functional group that bears an anionic charge, where the monomers J and L are monomers that are compatible to form a random copolymer; and
(ab) adding an initiator to the mixture to form a reaction mixture and allowing the reaction mixture to age for a period of time to provide the composite material, wherein
the monomers J and L belong to different monomer classes with differing polymerisation kinetics.

18. The composite material according to claim 1, wherein the monomer classes are acrylamide, acrylate and methacrylate.

19. The composite material according to claim 1, wherein the random copolymeric material is a poly(cationic acrylamide-ran-anionic methacrylate).
